(19) United States
(12) Patent Application Publication
(10) Pub. No.: US 2015/0150502 A1
(43) Pub. Date: Jun. 4, 2015

(54) INTEGRATED DEVICE FOR BREAST DISEASE DIAGNOSIS AND TREATMENT

(71) Applicant: Shiming WU, (US)

(72) Inventor: Shiming Wu, Chongqing (CN)

(21) Appl. No.: 14/406,751

(22) PCT Filed: Oct. 22, 2012
(86) PCT No.: PCT/CN2012/083278

§ 371 Date: Dec. 9, 2014

(30) Foreign Application Priority Data
Oct. 7, 2012 (CN) 201210376579.2

(51) Int. Cl. A61B 5/00; A61B 5/01; A61B 8/08; A61M 37/00

(52) U.S. Cl. CPC A61B 5/4839; A61M 37/0092; A61B 5/0075; A61B 5/015; A61B 5/0091; A61B 8/0825; A61B 5/0035; A61B 5/7425; A61B 5/7264; A61B 5/7282; A61B 5/725; A61B 5/7246; A61B 5/0013; A61B 5/0022; A61B 5/7405; A61B 8/5246

(57) ABSTRACT

The present invention discloses an integrated device for breast disease diagnosis and treatment, which includes a control host system, and a thermalgraphy subsystem, an ultrasoudimaging subsystem and a phonophoresis subsystem respectively in communication connection with the control host system. A display terminal is electrically connected to the control host system, and the control host system further has an intelligent interpretation expert system. The thermalgraphy subsystem is configured to perform infrared detection on breast to find an abnormal area; the ultrasoudimaging subsystem is configured to display structural changes of the abnormal area; the intelligent interpretation expert system is configured to perform a rational diagnosis and treatment scheme according to the detection results, in combination with a prestored image feature knowledge base of breast diseases; and the phonophoresis subsystem is configured to perform target drug permeation therapy according to a prompt of the intelligent interpretation expert system.

### FIELD OF THE TECHNOLOGY

The present invention relates to the technical field of medical apparatuses, and in particular, to a thermalgraphy ultrasoudimaging phonophoresis (TUP) integrated device for breast disease diagnosis and treatment.

### BACKGROUND

Breast diseases are common clinical and frequently-occurring diseases, and among the patients, female is in the majority. With the accelerating of the pace of modern urban life, women face heavy pressure of work and life at the same time, followed by neglect of their own health status. Presently, the incidence of breast diseases in the world is gradually increasing. According to the investigation and statistics of China Breast diseases Prevention Association, the incidence of female breast diseases has been up to 80%, if not being treated in time or being improperly treated, cancerization may occur, leading to life-threatening at any time. Therefore, breast diseases are also referred to as “pre-mastocarcinoma”.

Presently, there is no good way to prevent breast diseases especially breast cancer, early diagnosis is the most effective way to reduce the mortality rate and increase the cure rate for breast cancer. Palpation is the most convenient, the most direct, and the most economical examination method of breast diseases, and experienced specialists can touch breast lumps, know the size, the smoothness, the hardness and the activity of the lumps, and obtain the first-hand information through palpation. However, some lumps are small and deep set, and cannot be touched by hands, and some early-stage breast cancer has no lump formed, and cannot be determined by hands.

With the development of medical thermalgraphy, using the thermalgraphy in breast disease diagnosis has been accepted worldwidely, and has been certificated by the Food and Drug Administration (FDA) of USA. The thermalgraphy has the advantages of being non-invasive, painless and high sensitive, but has poor specificity. Presently, ultrasonic diagnosis has been widely used in breast disease diagnosis, has high specificity for structural changes in diagnosis, and the accuracy rate is up to 58% or more, but as for lesions, ultrasonic diagnosis is lack of sensitivity for lesions which is merely at the stage of functional changes, and has tiny structural changes. Presently, the thermalgraphy and the ultrasonic diagnosis are both used alone in breast disease diagnosis, and belong to different departments of a hospital and are rarely used in combination, thus causing great inconvenience for doctors and patients; and moreover, the thermalgraphy and the ultrasonic diagnosis are underused, and lack of correctness. Furthermore, the existing breast disease diagnosis method greatly depends on the doctor's experience and expertise, and is greatly influenced by subjective factors; therefore, missed diagnosis and misdiagnosis are easily caused, especially in general survey of breast diseases, and the efficiency and accuracy of diagnosis cannot be ensured.

### SUMMARY

An objective of the present invention is to provide an integrated device for breast disease diagnosis and treatment, so that one device of the present invention is capable of simultaneously realizing non-invasive and painless accurate diagnosis and accurate treatment of breast diseases, and providing great conveniences for both doctors and patients.

In order to achieve the above objective, the present invention provides an integrated device for breast disease diagnosis and treatment, which includes a control host system, and a thermalgraphy subsystem, an ultrasoudimaging subsystem and a phonophoresis subsystem respectively in communication connection with the control host system. A display terminal is electrically connected to the control host system, and the control host system further has an intelligent interpretation expert system. The thermalgraphy subsystem is configured to perform infrared detection on breast to find an abnormal area; the ultrasoudimaging subsystem is configured to display structural changes of the abnormal area; the intelligent interpretation expert system is configured to perform a rational diagnosis and treatment scheme according to the detection results of the thermalgraphy subsystem and the ultrasoudimaging subsystem, in combination with a prestored breast disease image feature knowledge base; and the phonophoresis subsystem is configured to perform target drug permeation therapy according to a prompt of the intelligent interpretation expert system.

The control host system includes a control mainframe and a plurality of communication interfaces, and the thermalgraphy subsystem, the ultrasoudimaging subsystem, the phonophoresis subsystem and the display terminal are respectively in communication connection with the control mainframe through corresponding communication interfaces; and the control mainframe further has an Ethernet interface for remote diagnosis

In the present invention, the control host system further prestores patient's information, a thermograph detected by the thermalgraphy subsystem, an ultrasonogram detected by the ultrasoudimaging subsystem, and result information of palpation of breast disease experts are stored in the control host system. The intelligent interpretation expert system performs the rational diagnosis and treatment scheme according to the patient's information, the thermograph, the ultrasonogram and the result information of palpation, in combination with a prestored image feature knowledge base of breast diseases.

Furthermore, the thermalgraphy subsystem includes a medical infrared thermograph, the medical infrared thermograph includes an optical system, a probe and control circuit, an acquisition system, a pretreatment system and an analysis system that are connected in sequence; and the optical system includes a wide-angle infrared lens, the probe and control circuit adopts an uncooled focal plane probe from FLIR of USA, the acquisition system includes an A/D converter, and the pretreatment system includes an FPGA infrared acquisition correction filter module.

After human infrared radiation is imaged on a photosensitive surface of the uncooled focal plane probe through the optical system, a serial analog image signal is output; the acquisition system is configured to perform analog-to-digital conversion on the signal, and a resulting signal is output by the A/D converter; the pretreatment system is configured to perform non-uniformity correction, filtering, temperature algorithm processing and Ethernet output operations on the image signal output by the A/D converter; and the analysis system is configured to perform temperature correction, image processing, image comparison, temperature analysis, report printing and medical record database management on the image data input by the pretreatment system.

In the present invention, the ultrasoudimaging subsystem includes a power module, an Ethernet module, a digital processing module and a probe interface module. One end of the

Ethernet module is electrically connected to the power module, and the other end is in communication connection with the digital processing module through a data bus. The probe interface module is electrically connected to the power module and the digital processing module respectively.

The digital processing module includes a digital signal processing module and a real-time control module electrically connected to the digital signal processing module. The digital signal processing module is further electrically connected to a digital beamformer module and an A/D conversion circuit in sequence. The real-time control module is further electrically connected to a transmission control circuit and a TGC circuit. The digital beamformer module and the transmission control circuit are electrically connected. The probe interface module includes two probe sockets, two switch arrays respectively electrically connected to the two probe sockets correspondingly, a probe selection module respectively electrically connected to the two probe sockets and the two switch arrays correspondingly, a transmitting and receiving circuit electrically connected to the two switch arrays, and a pulse generating circuit and an amplification circuit respectively electrically connected to the transmitting and receiving circuit. The two switch arrays, the transmitting and receiving circuit, the pulse generating circuit and the amplification circuit are respectively electrically connected to the power module.

Specifically, the pulse generating circuit is further electrically connected to the transmission control circuit of the digital processing module; the amplification circuit includes a first amplification circuit and a second amplification circuit electrically connected to the first amplification circuit. One end of the first amplification circuit is electrically connected to the transmitting and receiving circuit, and the other end is further electrically connected to the TGC circuit of the digital processing module. One end of the second amplification circuit is electrically connected to the power module, another end is electrically connected to the TGC circuit, and another end is further electrically connected to the A/D conversion circuit of the digital processing module.

In the present invention, the phonophoresis subsystem includes an ultrasonic conductivity meter, an ultrasonic conductive coupling electrode patch, an ultrasonic conductive coupling agent and a pharmaceutical preparation, the ultrasonic conductive coupling electrode patch is attached on human epidermis, the ultrasonic conductive coupling agent is filled between the ultrasonic conductive coupling electrode patch and the human epidermis, and the ultrasonic conductivity meter is connected to the electrode patch.

Additionally, the control host system includes a 3G wireless communication module and/or a wireless audio and video transceiver module.

According to the integrated device for breast disease diagnosis and treatment of the present invention with thermalgraphy, ultrasoudimaging and phonophoresis integrated, on the basis of a thermalgraphy device having high sensitivity and having functional diagnosis as the primary function and a ultrasonic diagnosis device having high specificity and having structural diagnosis as the primary function, in combination with palpation of breast disease experts, a large amount of test data is obtained, and in combination with other database data, an intelligent interpretation expert system is formed, and under the instruction of the intelligent interpretation expert system, a phonophoresis device delivers a targeted drug into target organ tissues in a non-invasive and painless way on the basis of correct target diagnosis, thereby realizing non-invasive and painless accurate diagnosis and accurate treatment of breast diseases, and providing great conveniences for both doctors and patients.

### BRIEF DESCRIPTION OF THE DRAWINGS

To illustrate the technical solutions according to the embodiments of the present invention or in the prior art more clearly, the accompanying drawings for describing the embodiments or the prior art are introduced briefly in the following. Apparently, the accompanying drawings in the following description are only some embodiments of the present invention, and persons of ordinary skill in the art can derive other drawings from the accompanying drawings without creative efforts.

FIG. 1 is a schematic module structural diagram of a specific embodiment of an integrated device for breast disease diagnosis and treatment of the present invention;

FIG. 2 is a schematic module structural diagram of a specific embodiment of a thermalgraphy subsystem in the present invention; and

FIG. 3 is a schematic module structural diagram of a specific embodiment of an ultrasoudimaging subsystem in the present invention.

### DETAILED DESCRIPTION

The technical solution of the present invention will be clearly and completely described in the following with reference to the accompanying drawings. It is obvious that the embodiments to be described are only a part rather than all of the embodiments of the present invention. All other embodiments obtained by persons skilled in the art based on the embodiments of the present invention without creative efforts shall fall within the protection scope of the present invention.

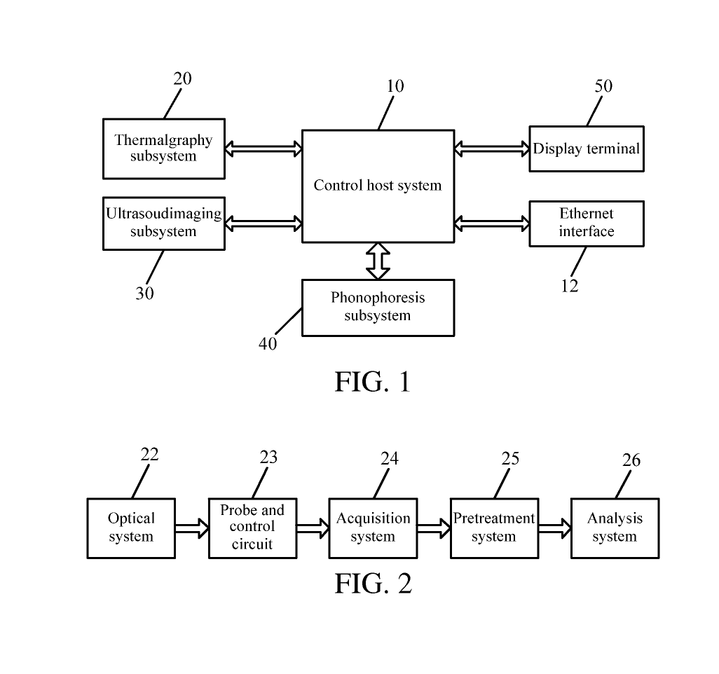

As shown in FIG. 1, the present invention provides an integrated device for breast disease diagnosis and treatment, which includes a control host system 10, and a thermalgraphy subsystem 20, an ultrasoudimaging subsystem 30 and a phonophoresis subsystem 40 respectively in communication connection with the control host system 10. A display terminal 50 is electrically connected to the control host system 10, and the control host system 10 further has an intelligent interpretation expert system (not shown). The thermalgraphy subsystem 20 is configured to perform infrared detection on breast to find an abnormal area; the ultrasoudimaging subsystem 30 is configured to display structural changes of the abnormal area; the intelligent interpretation expert system is configured to perform a rational diagnosis and treatment scheme according to the detection results of the thermalgraphy subsystem 20 and the ultrasoudimaging subsystem 30, in combination with a prestored breast disease image feature knowledge base; and the phonophoresis subsystem 40 is configured to perform target drug permeation therapy according to a prompt of the intelligent interpretation expert system. In the prior art, the thermalgraphy and the ultrasonic diagnosis belong to different departments of a hospital, and therefore, if patients with breast diseases merely choose one examination mode, it is difficult to ensure the diagnosis accuracy, and if patients with breast diseases choose both the two examination modes, they need to register at different departments and go to different departments for examination, which cannot ensure the efficiency of diagnosis. The integrated device for breast disease diagnosis and treatment of the present invention not only has thermalgraphy and ultrasonic diagnosis integrated, but also has diagnostic and therapeutic effects. Therefore, patients with breast diseases can finish thermalgraphy examination, ultrasonic diagnosis and target drug permeation therapy in one department at one time, thereby ensuring the efficiency and the accuracy of diagnosis and treatment at the same time.

In the present invention, the control host system 10 includes a control mainframe and a plurality of communication interfaces, and the thermalgraphy subsystem 20, the ultrasoudimaging subsystem 30, the phonophoresis subsystem 40 and the display terminal 50 are respectively in communication with the control mainframe through corresponding communication interfaces. As a preferred embodiment of the present invention, the control mainframe further has an Ethernet interface 12 for remote diagnosis. By means of the Ethernet interface 12, diagnosis and treatment information stored in the control mainframe can be remote-transmitted, so as to realize remote consultation and data sharing.

In the present invention, the control host system 10 further prestores patient's information, and the patient's information includes personal data of the patient and result information of other examination items. A thermograph detected by the thermalgraphy subsystem 20, an ultrasonogram detected by the ultrasoudimaging subsystem 30, and result information of palpation of breast disease experts are stored in the control host system 10. The intelligent interpretation expert system performs the rational diagnosis and treatment scheme according to the patient's information, the thermograph, the ultrasonogram and the result information of palpation, in combination with a prestored image feature knowledge base of breast diseases.

Furthermore, the thermalgraphy subsystem 20 (as shown in FIG. 2) includes a medical infrared thermograph, the medical infrared thermograph includes an optical system 22, a probe and control circuit 23, an acquisition system 24, a pretreatment system 25 and an analysis system 26 that are connected in sequence.

The optical system 22 is configured to receive infrared radiation of a detected object, and is formed by different infrared optical lenses according to requirements on the size of field of view and image quality, and functions in gathering, filtering and focusing the infrared radiation of the detected object. Specifically, the optical system 22 includes an infrared lens (not shown), and the infrared lens may be a wide-angle infrared lens. In order that the medical infrared thermograph has high temperature sensitivity, the infrared lens needs to have a relative aperture as large as possible and transmittance as high as possible. Optical focal length and aperture are main parameters of the optical system 22. The optical aperture determines the detection capability of the optical system 22, and is mainly determined by the radiation energy of the detected object and the operating distance and signal-to-noise ratio requirements of the optical system 22. Therefore, in the present invention, preferably, the angle of field of view of a lens having an aperture of 17 mm and an optical focal length f of 1 is 39°×30°.

In the present invention, the probe and the control circuit 23 adopts an uncooled focal plane probe from FLIR of USA. In the probe, vanadium oxide (VOx) is used as a thermistor material, the resistance thereof is approximately 100 Ω, and not only good noise performance but also low current noise are achieved, so that the thermalgraphy subsystem 20 can meet the requirements of high sensitivity, high resolution and high accuracy as much as possible. Furthermore, the acquisition system 24 of the present invention includes an A/D converter (not shown), and the A/D converter uses a 14-bit high-speed A/D converter to convert an output signal of the uncooled focal plane probe into a digital signal, for the pretreatment system 25 to process. The pretreatment system 25 includes an FPGA infrared acquisition correction filter module. After the human infrared radiation is imaged on a photosensitive surface of the uncooled focal plane probe by means of the optical system 22, a serial analog image signal is output. The acquisition system 24 is configured to perform analog-to-digital conversion on the signal, and a resulting signal is output by the A/D converter. The pretreatment system 25 is configured to perform non-uniformity correction, filtering, temperature algorithm processing and Ethernet output operations on the image signal output by the A/D converter. The analysis system 26 is configured to perform temperature correction, image processing, image comparison, temperature analysis, report printing and medical record database management on the image data input by the pretreatment system 25.

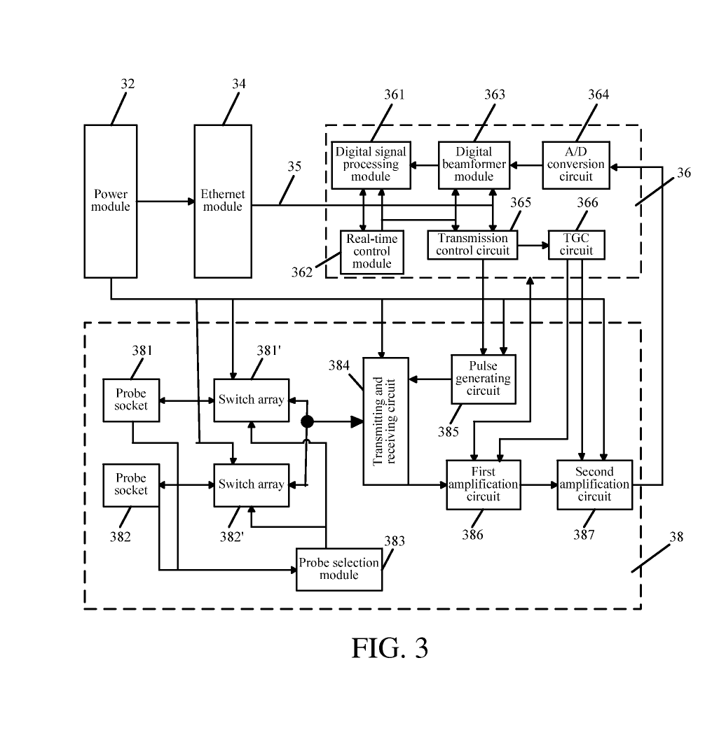

As shown in FIG. 3, the ultrasoudimaging subsystem 30 includes a power module 32, an Ethernet module 34, a digital processing module 36 and a probe interface module 38. One end of the Ethernet module 34 is electrically connected to the power module 32, and the other end is in communication connection with the digital processing module 36 through a data bus 35. The probe interface module 38 is electrically connected to the power module 32 and the digital processing module 36 respectively.

The digital processing module 36 includes a digital signal processing module 361 and a real-time control module 362 electrically connected to the digital signal processing module 361. The digital signal processing module 361 is further electrically connected to a digital beamformer module 363 and an A/D conversion circuit 364. The real-time control module 362 is further electrically connected to a transmission control circuit 365 and a time gain control (TGC) circuit 366, and the digital beamformer module 363 and the transmission control circuit 365 are electrically connected.

The probe interface module 38 includes two probe sockets 381 and 382, two switch arrays 381′ and 382′ respectively electrically connected to the two probe sockets 381 and 382 correspondingly, a probe selection module 383 respectively electrically connected to the two probe sockets 381 and 382 and the two switch arrays 381′ and 382′ correspondingly, a transmitting and receiving circuit 384 electrically connected to the two switch arrays 381′ and 382′, and a pulse generating circuit 385 and an amplification circuit respectively electrically connected to the transmitting and receiving circuit 384. The two switch arrays 381′ and 382′, the transmitting and receiving circuit 384, the pulse generating circuit 385 and the amplification circuit are respectively electrically connected to the power module 32. Specifically, the pulse generating circuit 385 is further electrically connected to the transmission control circuit 365 in the digital processing module 36. The amplification circuit includes a first amplification circuit 386 and a second amplification circuit 387 electrically connected to the first amplification circuit 386. One end of the first amplification circuit 386 is electrically connected to the transmitting and receiving circuit 384, the other end is further electrically connected to the TGC circuit 366 in the digital processing module 36. One end of the second amplification circuit 387 is electrically connected to the power module 32, another end is electrically connected to the TGC circuit 366, and anther end is further electrically connected to the A/D conversion circuit 364 of the digital processing module 36. Single thermalgraphy detection merely can find an abnormal breast area, but cannot display structural changes of the abnormal area, and the sensitivity and specificity are poor. The existing ultrasonic detection is superior in positioning and basic morphology display; however, it is not sensitive to early-stage small tumors. Therefore, if only one method is used to perform breast disease diagnosis, missed diagnosis and misdiagnosis are easily caused. On the basis that the thermalgraphy subsystem 20 finds the abnormal area through examination, the integrated device for breast disease diagnosis and treatment of the present invention performs ultrasonic diagnosis on the abnormal area. In the present invention, the power module 32 provides normal working power supply for the entire ultrasoudimaging subsystem 30. The digital processing module 36 sends a logic control signal to control, through the real-time control module 362 and the transmission control circuit 365, the pulse generating circuit 385 to generate a pulse signal. The transmitting and receiving circuit 384 adds a high-voltage pulse signal to a corresponding probe (not shown) according to the selection of the probe selection module 383, and the probe sends an ultrasonic probe signal. When an echo is generated on human tissues by the ultrasonic probe signal sent by the probe, the returned weak signal is fed to the transmitting and receiving circuit 384. The transmitting and receiving circuit 384 amplifies the echo signal through the amplification circuit, and then performs analog-to-digital conversion through the A/D conversion circuit 364. The digital signal after analog-to-digital conversion is fed to the digital beamformer module 363 for processing. The digital beamformer module 363 mainly functions to complete reception delay, dynamic focusing and dynamic apodization of the echo signal, that is, to perform delay and weighted summation on the received multi-channel ultrasonic echo signal. The digital beamformer module 363 transfers the signal after focusing to the digital signal processing module 361, and then transfers a detected B-ultrasonic image to the control host system 10 through the Ethernet module 34. A local gain adjustable circuit is added to the TGC circuit 366 in the present invention, so as to achieve the effect of enhancing an echo pulse or a burst signal on some designated areas, so that an operator can provide additional amplification for a signal of an area of any depth, thereby effectively improving the quality of image diagnosis.

Furthermore, the phonophoresis subsystem 40 in the present invention includes an ultrasonic conductivity meter, an ultrasonic conductive coupling electrode patch, an ultrasonic conductive coupling agent and a pharmaceutical preparation (not shown). The ultrasonic conductive coupling electrode patch is attached on human epidermis, the ultrasonic conductive coupling agent is filled between the ultrasonic conductive coupling electrode patch and the human epidermis, and the ultrasonic conductivity meter is connected to the electrode patch. The phonophoresis subsystem 40 is an electronic medical device for realizing needle-free injection and target delivery of drugs with ultrasound as the main driving force, uses the physical energy such as ultra-low-frequency ultrasound (20 KHz), and utilizes a focused radiation pressure of ultrasound to generate an impact, so as to convert mechanical energy into strong kinetic energy, thereby emitting a drug and delivering the drug into the human body through the skin, and achieving the effect of needle-free injection.

The integrated device for breast disease diagnosis and treatment of the present invention has thermalgraphy and jointly ultrasound-mediated phonophoresis integrated. In specific use, the thermalgraphy subsystem 20 first performs infrared detection, and after an abnormal area is found, the ultrasoudimaging subsystem 30 performs ultrasonic detection on the abnormal area. Through combination of the thermalgraphy subsystem 20 having high sensitivity and having functional diagnosis as the primary function and the ultrasoudimaging subsystem 30 having high specificity and having structural diagnosis as the primary function, not only early-stage small tumors can be detected and breast abnormalities can be effectively prompted, but also a qualitative conclusion can be provided for an abnormal or benign lump, cystic tumors and solid tumors can be effectively distinguished, and effective positioning and morphology display can be performed. A thermograph including information such as temperature distribution, metabolic state and distribution of blood vessels obtained by the thermalgraphy subsystem 20 through detection, and an ultrasonogram, including tissue structure information of an abnormal area in the thermograph, obtained by the ultrasoudimaging subsystem 30 through detection are transferred to the control host system 10. The intelligent interpretation expert system in the control host system 10 performs the rational diagnosis and treatment scheme according to the patient's information, the thermograph, the ultrasonogram and the result information of palpation (the size, the texture and the lymph node distribution of the lump obtained through feeling by hands), in combination with a large amount of prestored breast disease image feature knowledge base. The phonophoresis subsystem 40 directly sends a drug to the tissue lesion according to the diagnosis and treatment scheme prompted by the intelligent interpretation expert system, thereby realizing target accurate treatment. The phonophoresis in the prior art can deliver the drug into the target organ in a non-invasive and painless way; however, the efficacy is influenced due to a wrong target, and therefore, it is critical to find the correct target. In the present invention, the intelligent interpretation expert system combines a large number of results of thermalgraphy, ultrasonic images and palpation of experts and other basic knowledge in the database, the target can be accurately determined, and on the basis of accurate determination of the target, a targeted drug is delivered into the target organ tissues in a non-invasive and painless way, thereby realizing the efficacy of non-invasive and painless accurate diagnosis and accurate treatment.

Additionally, as an alternative embodiment of the present invention, a 3G wireless communication module and/or a wireless audio and video transceiver module (not shown) may be disposed in the control host system 10. Through the setting of the 3G wireless communication module, remote transmission, remote consultation and data sharing of diagnostic data in the wild or a moving state can be realized. Through the setting of the wireless audio and video transceiver module, the diagnostic data can be transferred wirelessly and input into virtual video glasses (not shown), so that a virtual image is formed in front of the human eyes, and the audio signal is converted into sound through a headset, thereby enabling multiple persons to observe the diagnostic image at the same time and realizing consultation of multiple persons. Not only the image and details can be observed clearly and conveniently, but also the problem of remote transmission, remote consultation and data sharing of diagnostic data in the wild or a moving state can be solved, so that on-site diagnosis can be used in remote consultation, training, teaching and other activities, thereby realizing close combination of medical treatment with training, teaching and other activities.

The above descriptions are merely preferred embodiments of the present invention, but not intended to limit the present invention. Any modification, equivalent replacement, or improvement made without departing from the spirit and principle of the present invention should fall within the scope of the present invention.

1. An integrated device for breast disease diagnosis and treatment, comprising
a control host system, and
a thermalgraphy subsystem, an ultrasoudimaging subsystem and a phonophoresis subsystem respectively in communication connection with the control host system; wherein
a display terminal is electrically connected to the control host system, and the control host system further comprises an intelligent interpretation expert system;
the thermalgraphy subsystem is configured to perform infrared detection on breast to find an abnormal area;
the ultrasoudimaging subsystem is configured to display structural changes of the abnormal area;
the intelligent interpretation expert system is configured to perform a rational diagnosis and treatment scheme according to detection results of the thermalgraphy subsystem and the ultrasoudimaging subsystem, in combination with a prestored breast disease image feature knowledge base; and
the phonophoresis subsystem is configured to perform target drug permeation therapy according to a prompt of the intelligent interpretation expert system.

2. The integrated device for breast disease diagnosis and treatment according to claim 1, wherein the control host system comprises a control host and a plurality of communication interfaces, and the thermalgraphy subsystem, the ultrasoudimaging subsystem, the phonophoresis subsystem and the display terminal are respectively in communication connection with the control host through corresponding communication interfaces; and the control host further comprises an Ethernet interface for remote diagnosis.

3. The integrated device for breast disease diagnosis and treatment according to claim 2, wherein the control host system prestores patient's information; a thermograph detected by the thermalgraphy subsystem, an ultrasonogram detected by the ultrasoudimaging subsystem, and result information of palpation of breast disease experts are stored in the control host system; and the intelligent interpretation expert system performs the rational diagnosis and treatment scheme according to the patient's information, the thermograph, the ultrasonogram and the result information of palpation, in combination with a prestored image feature knowledge base of breast diseases.

4. The integrated device for breast disease diagnosis and treatment according to claim 3, wherein the thermalgraphy subsystem comprises a medical infrared thermograph, the medical infrared thermograph comprises an optical system, a probe and control circuit, an acquisition system, a pretreatment system and an analysis system that are connected in sequence; and the optical system comprises a wide-angle infrared lens, the probe and control circuit adopts an uncooled focal plane probe from FUR of USA, the acquisition system comprises an A/D converter, and the pretreatment system comprises an FPGA infrared acquisition correction filter module.

5. The integrated device for breast disease diagnosis and treatment according to claim 4, wherein after human infrared radiation is imaged on a photosensitive surface of the uncooled focal plane probe through the optical system, a serial analog image signal is output; the acquisition system is configured to perform analog-to-digital conversion on the signal, and a resulting signal is output by the A/D converter; the pretreatment system is configured to perform non-uniformity correction, filtering, temperature algorithm processing and Ethernet output operations on the image signal output by the A/D converter; and the analysis system is configured to perform temperature correction, image processing, image comparison, temperature analysis, report printing and medical record database management on the image data input by the pretreatment system.

6. The integrated device for breast disease diagnosis and treatment according to claim 3, wherein the ultrasoudimaging subsystem comprises a power module, an Ethernet module, a digital processing module and a probe interface module; one end of the Ethernet module is electrically connected to the power module, and the other end is in communication connection with the digital processing module through a data bus; and the probe interface module is electrically connected to the power module and the digital processing module respectively.

7. The integrated device for breast disease diagnosis and treatment according to claim 6, wherein the digital processing module comprises a digital signal processing module and a real-time control module electrically connected to the digital signal processing module, the digital signal processing module is further electrically connected to a digital beamformer module and an A/D conversion circuit in sequence, the real-time control module is further electrically connected to a transmission control circuit and a TGC circuit, and the digital beamformer module and the transmission control circuit are electrically connected; the probe interface module comprises two probe sockets, two switch arrays respectively electrically connected to the two probe sockets correspondingly, a probe selection module respectively electrically connected to the two probe sockets and the two switch arrays correspondingly, a transmitting and receiving circuit electrically connected to the two switch arrays, a pulse generating circuit and an amplification circuit respectively electrically connected to the transmitting and receiving circuit; the two switch arrays, the transmitting and receiving circuit, the pulse generating circuit and the amplification circuit are respectively electrically connected to the power module.

8. The integrated device for breast disease diagnosis and treatment according to claim 7, wherein the pulse generating circuit is further electrically connected to the transmission control circuit of the digital processing module; the amplification circuit comprises a first amplification circuit and a second amplification circuit electrically connected to the first amplification circuit, one end of the first amplification circuit is electrically connected to the transmitting and receiving circuit, and the other end is further electrically connected to the TGC circuit of the digital processing module; and one end of the second amplification circuit is electrically connected to the power module, another end is electrically connected to the TGC circuit, and another end is further electrically connected to the A/D conversion circuit of the digital processing module.

9. The integrated device for breast disease diagnosis and treatment according to claim 3, wherein the phonophoresis subsystem comprises an ultrasonic conductivity meter, an ultrasonic conductive coupling electrode patch, an ultrasonic conductive coupling agent and a pharmaceutical preparation, the ultrasonic conductive coupling electrode patch is attached on human epidermis, the ultrasonic conductive coupling agent is filled between the ultrasonic conductive coupling electrode patch and the human epidermis, and the ultrasonic conductivity meter is connected to the electrode patch.

10. The integrated device for breast disease diagnosis and treatment according to claim 1, wherein the control host system comprises a 3G wireless communication module and/or a wireless audio and video transceiver module.

11. The integrated device for breast disease diagnosis and treatment according to claim 2, wherein the control host system comprises a 3G wireless communication module and/or a wireless audio and video transceiver module.

12. The integrated device for breast disease diagnosis and treatment according to claim 3, wherein the control host system comprises a 3G wireless communication module and/or a wireless audio and video transceiver module.

13. The integrated device for breast disease diagnosis and treatment according to claim 4, wherein the control host system comprises a 3G wireless communication module and/or a wireless audio and video transceiver module.

14. The integrated device for breast disease diagnosis and treatment according to claim 5, wherein the control host system comprises a 3G wireless communication module and/or a wireless audio and video transceiver module.

15. The integrated device for breast disease diagnosis and treatment according to claim 6, wherein the control host system comprises a 3G wireless communication module and/or a wireless audio and video transceiver module.

16. The integrated device for breast disease diagnosis and treatment according to claim 7, wherein the control host system comprises a 3G wireless communication module and/or a wireless audio and video transceiver module.

17. The integrated device for breast disease diagnosis and treatment according to claim 8, wherein the control host system comprises a 3G wireless communication module and/or a wireless audio and video transceiver module.

18. The integrated device for breast disease diagnosis and treatment according to claim 9, wherein the control host system comprises a 3G wireless communication module and/or a wireless audio and video transceiver module.
